(19) United States
(12) Patent Application Publication
(10) Pub. No.: US 2007/0231353 A1
(43) Pub. Date: Oct. 4, 2007

(54) Compositons and methods for maintaining eyelid hygiene

(75) Inventors: Jeffrey P. Gilbard, Weston, MA (US); Ed V. Breen, Manchester City, VT (US)

Correspondence Address: EDWARDS ANGELL PALMER & DODGE LLP, P.O. BOX 55874, BOSTON, MA, 02205, US

(73) Assignee: Advanced Vision Research, Inc., Woburn, MA (US)

(21) Appl. No.: 11/529,096

(22) Filed: Sep. 27, 2006

Related U.S. Application Data
(63) Continuation of application No. PCT/US05/10881, filed on Mar. 31, 2005.
Application No. PCT/US05/10881 is a continuation of application No. 10/816,513, filed on Mar. 31, 2004.

(51) Int. Cl. A61K 9/12

(52) U.S. Cl. 424/400

(57) ABSTRACT

The present invention is directed to novel compositions and methods effective for maintaining eyelid hygiene, e.g., therapeutic treatment and prophylaxis. The present invention further provides cleanser compositions and methods that are compliance-enhancing and useful for daily prophylaxis involving the easy and safe application directly to the eyelid in controlled doses for maintenance of eyelid hygiene. The cleanser compositions of the present invention can be in the form of a foam.

### BACKGROUND OF THE INVENTION

“Dry eye” is the world's most common eye disease. “Dry eye” indicates the lack of quantity and/or quality of the tear film. It is described as epidemic and pandemic.

The tear film is a dynamic unit composed of an inner, middle and outer layer. The combined three layers are approximately 3 microns in total thickness, wherein a slight change in the composition of any part of the unit will cause the entire tear film to rapidly deteriorate. A lack of tears leads to a lack of nutrients and oxygen to the cornea.

Manifestations of dry eye may initially occur as ‘discomfort’ in the eye, including pain from irritation, sandy gritty sensation, loss of night vision, and excessive watery eyes, and often results in contact lens drop out, or LASIK surgery dissatisfaction. When left untreated, the consequences of dry eye can be severe, and even result in loss of vision (e.g., from desiccation of the corneal epithelium, ulceration and perforation of the cornea, or an increased incidence of infectious disease).

There are two main causes for dry eye.

1) A lack of tear production (“tear-deficiency dry eye”).

Tear-deficiency dry eye can be further classified as related to the “Sjögren syndrome” or “Non-Sjögren”. Sjögren syndrome is a chronic disease in which white blood cells attack the moisture-producing glands, including those that produce tear film. The non-Sjögren syndrome related type of dry eye syndromes are those where the eye does not produce tears because of disorders of the tear gland; for example, vitamin A deficiency, contact lens wear, and diabetes and eye infections.

2) An increase in evaporation of the tear film (“evaporative dry eye”).

The primary manifestation of evaporative dry eye is a dysfunction of the outer, lipid layer. Normally, evaporation of the entire tear film is prevented by the thin, oily, outer layer, which protects the ‘watery’ parts of the overall tear film. This sub-micron lipid layer floats on top of the entire watery layer of the tear film as 1/70^{th} of the total 3 microns, is similar in thickness to a sub micron film of a soap bubble and also functions similarly. Puncture a soap bubble and the air will escape. A dysfunction of the lipid layer causes the tear to evaporate, to ‘break up”. This can be measured in seconds and is called tear break up time (“TBUT”). This critical, but extremely thin and fragile, lipid layer is produced by tiny glands located outside the eye, in the skin of the eyelid.

The primary causes of the dysfunction of the lipid layer are all associated with inflammation (e.g., anterior and posterior blepharitis/Meibomian gland dysfunction, hordeolum, sty, and rosacea).

The etiological factors of all these inflammations are an overgrowth of bacteria (and parasites) and their toxic waste. These bacteria not only cause the lipid tear film to dysfunction, but they also destroy and block the very lipid-producing infrastructure by attacking the glands at their roots in the eyelid skin. Unfortunately, the particular types of bacteria and parasites that cause the inflammation/infections are common. The chance of having these on the eyelids is near 100%. In themselves, they are not dangerous, but it is the overgrowth and their toxic waste on the lid margin and the eyelashes that must be avoided. Allowing the bacteria and parasites to fester, nest and proliferate must be prevented, especially if one is diagnosed as a dry eye sufferer, or if there is an increased risk of having dry eye symptoms such as LASIK patients, and contact lens wearers.

With of dry eye exacerbated by bacterial infections, it is therefore no surprise that critical (daily or twice daily) eyelid hygiene is prescribed by physicians and recommended by health institutions worldwide. (For the life of the patient, because, as mentioned, dry eye is chronic, with no known cure and will only worsen with age).

And herein lies the problem. People tend to wash their face, but not their eyelids. Even though daily or twice daily eyelid hygiene is so critical, there is no compliance. Due to a lack of alternatives, typically “baby shampoo” is recommended. This current ‘prescription” for eye hygiene has significant non-compliance issues. For example, practical applications of baby shampoo are confused by the numerous questions that remain unanswered with respect to effective use of the shampoo as a treatment for eyelid disorders. By way of illustration, how much water is to be added, when and how to apply, and how often is safe to apply? Clearly there is no standard dosage for this ‘treatment’. Thus, the baby shampoo is mixed with cocktails of warm or hot water in many different ratios, and the solution is then applied with non-sterile, dubious applicators ranging from slicks with cotton tips to unsanitary wash cloths. Thus not only endangering the patient, but also virtually ensuring little, if any, compliance.

Also available are expensive commercial “eyelid scrubs”. These commercial eyelid “scrubs” can come in two forms; either impregnated, pre-moistened towelettes/pads, or as bottled cleansers. Bottled cleansers are applied to a non-sterile applicator and are then applied in undefined, non-standardized dosage foams created by the agitation of the applicator infused with a cleanser composition.

With only these “alternatives”, it is not surprising that there is little or no compliance to eyelid hygiene. It is further evidenced by way of commercial sales for eye care products, which shows a clear indication that people simply do not clean their eyelids: In contrast to the US eye care market for eye drops (excluding contact lens solutions) of $1 billion, current eyelid scrubs show less than $8 million in retail sales.

In addition to compliance issues, the existing cleanser compositions often overlook the special characteristics of the periocular skin. The periocular skin is more penetrable, is thinner, and is more fragile than other parts of the skin, and is in constant motion. In fact, the eyelid scrubs are typically high pH solutions and are detrimental to the periocular skin on a regular basis, because they could create further pathways for pathogens. As such, the eyelid scrubs can be detrimental to the skin, not just because they are abrasive to the skin through their applicator, but also through their composition, if used on a daily, high frequency basis for treatment of ocular disorders.

Thus, the exacerbating factors of the most common eye disease, dry eye, is the overgrowth on the eyelid of common bacteria and parasites as well as their toxic waste. Not only do these organisms interfere with the function of the tear film, they destroy the tear-producing infrastructure. Since dry eye has no known cure, optimization of the infrastructure is critical. For that reason it is deemed vital to continuously prevent any festering and proliferation of bacteria and parasites on the eyelid. Daily or twice daily eyelid hygiene is prescribed as critical. However, due to a lack of a simple method and composition there is very little compliance and thus, this need has not bee addressed.

### SUMMARY OF THE INVENTION

At present, there exists a need for a simple and easy method of cleaning an eyelid that provides the correct and defined dosage in the form of a foam, with no significant margin of error, and reduced risk of poking of the eye, e.g., with towelettes, Q-tips, or washcloths.

The present invention is directed to novel compositions and methods effective for maintaining eyelid hygiene, e.g., therapeutic treatment and prophylaxis. The methods and compositions disclosed herein are compliance-enhancing and useful for daily prophylaxis. These methods involve the easy and safe application of controlled foam directly to the eyelid in controlled doses effective for maintenance of eyelid hygiene. Thus, disruption of the delicate top layer of periocular skin around the eye by application of an undefined foam cleanser composition to the eye is avoided, as well as the need for dangerous applicators that could poke, scratch, or infect the eye. In this way, the present methods represent significant steps for increased compliance in the daily maintenance of eyelid hygiene.

Definitions

The invention will be described with reference to following definitions that, for convenience, are collected here.

The term “controlled concentration” is defined as a characteristic of a mixture where the ratio of active ingredient(s) to non-active ingredient(s) is controllable at a prescribed level, and therefore definitive amounts of the mixture, and ingredients contained therein, can be delivered/distributed. Such a characteristic is useful in providing controllable dosage regimens (i.e., improving predictability of the dose delivered).

The term “controlled concentration foam” is defined as a foam formulated as a controlled concentration mixture of active ingredient to non-active ingredients, e.g., deionized water. The controlled concentration foam is in contrast to a liquid solution that requires further preparation, e.g., dilution and/or agitation to create a foam prior to application to the eyelid.

The term “cleaning an eyelid” is used herein to describe the act of significantly reducing the amount of dirt, debris, or otherwise undesired material, e.g., bacteria, from an eyelid.

The term “direct application” is used herein to describe the application of a cleanser composition to a subject, e.g., an eyelid of a subject, with no additional processing or preparation of the cleanser, e.g., no manual foaming or lathering, prior to application to the eyelid.

The term “dispensing” is defined as the act of delivering a cleanser composition to an applicator that has not been stored in direct contact with an applicator, e.g., in contrast to commercially available eyelid scrubs where the sponge is stored in direct contact with the cleanser liquid.

The term “dry eye” is known in the art as a condition of a subject that has a lack of quality and/or quantity of tears. Dry eye is often an age related disease. Meibomian gland dysfunction is the most frequent cause of dry eye and manifests itself in such forms as encrustation of the eyelid margins, sty, hordeolum or other inflammation of the connective tissue. Meibomian gland dysfunction is commonly linked with ocular rosacea, blepharitis, and other inflammation of the eyelids. All of these causes of inflammations of the skin are related to bacterial infection.

The term “eyelid” as used herein, includes the ocular surface, both the interior and exterior surfaces of the eyelid, the eyelid margin, the glands in and around the eyelid margins, the hair follicles of the eye, the eyelashes, and the periocular skin surrounding the eye. A front and side expanded view of the eyelid is shown in FIGS. 1A and 1B, respectively.

The term “eyelid disorder” is defined as a disorder that results in inflammation of tear producing glands or inflammation of the lipid producing glands that are located in the eyelid. Exemplary eyelid disorders include, but are not limited to proptosis, ectropion, entropion, incomplete blinking, pterygia pingueculae, conjunctivochalasis, encrustation of the eyelid margins, sty, hordeolum or other inflammation of the connective tissue, and nocturnal lagophthalmos.

The term “localized and sustained massaging”, as used herein, defines a manner of agitation of an eyelid of a subject. The massaging is focused on the eyelid for an amount of time sufficient for cleaning an eyelid, and results in significant agitation of the glands of the eyelid. This term is distinguishable from the incidental agitation of the eyelid associated with, for example, washing the entire face including the eyelid. In certain embodiments, the massaging is sustained for at least 5 seconds and possible for 10-30 seconds.

The term “ocular disorder” as used herein, includes ocular surface disorders, periocular skin disorders, and eyelid disorders, and particularly includes dry eye and symptoms related thereto. Exemplary ocular disorders include, but are not limited to dysfunctions of the tear film, inflammation of the eyelid dermis.

The term “sponge” as used herein includes all absorbent materials such as pads, swabs, tissues, Q-tips, washcloths, or fiber applicators of any kind that may be used to induce foaming and/or used as an applicator for an eyelid cleanser.

The term “transiently stable foam” is used herein to define a foam that maintains its foam nature for a sufficient amount of time as to be useful in the application to an eyelid of a subject. A transiently stable foam need not be present in the form of a foam indefinitely, but rather only as long as needed to provide a subject sufficient time to apply the dispensed foam to the eyelid.

The term “treatment” as used herein is defined as prophylactic treatment (e.g., daily preventative use) or therapeutic treatment (e.g., a single treatment or a course of treatment) of a subject with an ocular disorder, which results in the reduction, alleviation, or elimination of at least one symptom of an ocular disorder.

Methods and Compositions

Maintaining the health of the eyelid and surrounding tissue is a critical step in improving the function of the tear and lipid producing glands of the eyelid. In fact, it is not the type of bacteria, nor the quantity, but their ability to penetrate skin that causes ocular problems. Healthy skin is less penetrable by infection or infestation. FIG. 2 shows the entrance of bacteria through a skin portal, e.g., a follicle. As such, the present invention is intended to emphasize the maintenance of eyelid hygiene through prophylaxis in addition to treatment using the compositions and methods of the present invention. The present methods, which involve localized and sustained massaging of the eyelids, assist in the removal of any overgrowth of common bacteria and parasites, including the removal of the excrement released by these organisms that frequently causes the dysfunction of the tear producing glands as well as infection and inflammation of part of the eyelid (i.e., which in turn can further accelerate the cycle of tear film dysfunction).

Accordingly, the invention is directed to a cleanser composition in the form of a controlled concentration foam and it is suitable for direct application to an eyelid of a subject effective for maintaining eyelid hygiene. The foam may be generated in a substantially sponge free environment. In addition, the cleanser composition may be specifically formulated for the treatment of an ocular disorder, e.g., an ocular disorder selected from inflammation of tear producing glands or inflammation of the lipid producing glands.

The invention is further directed to a method of cleaning an eyelid of a subject or to treat ocular disorder in a subject. The method comprises the steps of providing a dispensing apparatus containing a cleanser composition, dispensing a controlled concentration of eyelid cleanser composition from the dispensing apparatus in the form of a transiently stable foam, applying the foam to the eyelid, and agitating the eyelid by localized and sustained massage of eyelid foam onto the eyelid. The foam maybe dispensed onto a fingertip and the fingertip is used to agitate the eyelid. The subject in need of treatment may have been diagnosed previously.

Furthermore, the dispensing apparatus may deliver the transiently stable foam to an applicator, e.g., a finger, utilizing a pump mechanism or a squeeze mechanism. It would also be understood by the ordinarily skilled artisan that the methods described above may utilize the controlled concentration foam in combination with any mechanically abrasive cleaning technique, for example, commercially available eyelid scrubs.

The cleaning agent of the cleanser composition may be any aqueous solution that can be formulated to form a transiently stable foam in a controlled concentration, provided that the composition is not significantly deleterious to the comfort or health of the eye and/or detracts from the compliance of use. For example, the cleanser composition may be an aqueous formulation formulated with sufficient additives to produce transiently stable foam from a dispenser engineered to produce a controlled concentration foam.

The periocular skin is distinct in nearly every important function from the rest of the body and requires special attention, especially since it is the first to manifest age related changes. At the same time, this distinct periocular skin also provides accommodation for most all of the important tear producing glands that are responsible for the tear film. (e.g., the Meibomian glands, e.g., the glands of Zeiss, Moll, and Kraus). Therefore, the present cleanser composition preferably is formulated to account for the distinct characteristics of the periocular skin. These characteristics include, but not limited to, the lower lipid count, the increased fragility as compared with other areas of skin, the lower layer count in the corneum stratum, the higher rate of exfoliation, the warm and moist environment which is conducive to infection, the age-related changes, and the fact that it represents the area where critical tear producing glands are located.

The cleanser composition may contain the following components:
an anti-inflammatory agent in the range of about 0.01 to 7.7% by weight, selected from the group consisting of zwitterionic organic compound derivatives, e.g., ether analogs of N-(2-hydroxyl ethyl) piperazine N′ (12-propane sulfonic acid) (HEPES), urethane derivative of HEPES, or aliphatic esters synthesized from piperazine-N′ propane sulfonic acid, e.g., an ester of HEPES selected from the group consisting of acetate ester, oleate ester, and linoleate ester or a long chain aliphatic ester of HEPES;
a pH-control agent and antioxidant agent in the range about 0.05-7.5% by weight selected from the group consisting of a carboxylic acid derivative of propane;
a first tissue healing agent in the range of about 0.01 to 1% by weight selected from the group consisting of allantoin and panthenol;
a first water soluble surfactant present in the range of about 0.02-20% by weight, eyelid first surfactant selected from the group consisting of sodium laureth sulfate, potassium lauryl phosphate polysorbate 60, potassium tridecyl phosphate polysorbate 60, potassium lauryl phosphate and potassium tridecyl phosphate, e.g., sodium laureth sulfate in the range of about 0.3-20% by weight;
a second water soluble surfactant in the range of about 0.01 to 7.0% by weight, which also aids in lipid replacement selected from the group consisting of di sodium lauroamphodiacetate and linoleamidopropyl PG-diammonium chloride phosphate;
a skin conditioning and antibacterial agent comprising a phospholipid essential fatty acid in the range of about 0.1 to 5.0% by weight;
a microbiological preservative present in the range of about 0.01-5.7% by weight and selected from the group consisting of phenoxyethanol and diazolidinyl urea propylene glycol/methyl-propyl parabens; and
a viscosity regulating agent in the range of about 0.1 to 6% by weight selected from one of the alkali metal salts of hydrochloric acid.

The composition may further comprise a solubilizer in the range of about 0.01 to 5% by weight, e.g., a polyoxyethylene derivative of a fatty acid ester of sorbitol, and/or a second preservative selected from the group consisting of diazolidinyl urea propylene glycol, and methyl-propyl paraben, and/or a third surfactant of sodium borage-amidopropyl hydroxyphosphate.

The present composition may employ HEPES derivatives which are pharmacologically active as anti-phospholipase and anti-inflammatory compounds, specifically where the active ingredients are certain long chain esters of selected zwitterionic compounds based-on N-substituted taurine, e.g., aliphatic esters of HEPES. Generally, the useful HEPES derivatives of the invention may be produced by catalytically reacting an alkali metal salt of HEPES with an alkyl-substituted, saturated or unsaturated, aliphatic salt, such as methyl oleate, methyl linoleate, methyl palmitate, methyl stearate, methyl myristate, and methyl behenate. The reactants are reacted in equimolecular amounts, carried out either with or without a non-aqueous solvent, such as acetone, and at a temperature range of 0° C. to the chosen solvent's reflux temperature.

The purification of the crude ester is carried out by means of crystallization in an organic solvent, dissolved in methanol, and recrystallized. U.S. Pat. No. 6,114,337, granted Sep. 5, 2000, provides detailed synthesis examples of the zwitterionic organic compounds useful here, including ether analogs and urethane derivatives of HEPES.

In another embodiment, the present invention provides a cleanser composition having an anti-inflammatory agent, a pH-control agent and antioxidant agent, a tissue healing agent, a soap containing one or more surfactants, a water-soluble surfactant, a skin conditioning and antibacterial agent, a microbiological preservative system, an osmolality agent and deionized water. The cleanser composition can further include a fragrance such as an aqueous extract of chamomile. The osmolality of the solution of the cleanser composition can be less than about 200 mOsmol/kg., preferably about 150 mOsmol/kg. The cleanser composition is preferably, though not necessary, in the form of a controlled foam.

The anti-inflammatory agent can be present in a range of about 0.01 to 7.7% by weight of the composition. The anti-inflammatory agent can include zwitterionic organic compound derivatives synthesized from piperazine-N′ propane sulfonic acid, such as Hepes acetate.

The pH-control agent and antioxidant agent is present in a range of about 0.05-7.5% by weight of the composition. The preferred pH-control agents are carboxylic acid derivatives of propane, citric acid or a mixture thereof.

The tissue healing agent can be present in a range of about 0.01 to 1% by weight of the composition. Examples of the healing agent are, but are not limited to, allantoin, panthenol and mixtures thereof.

The cleanser composition contains a soap, in the form of a water-soluble surfactant, in a range of about 0.02-20% by weight of the composition. The soap can contain one or more of sodium lauryl sulfate, potassium lauryl phosphate, polysorbate 60 (polyoxyethylene (20) sorbitan monostearate), polysorbate 80 (polyoxyethylene sorbitan monooleate), sodium borageamidopropyl hydroxyphosphate, potassium tridecyl phosphate, potassium monoalkyl phosphate, PEG-80, sorbitan laurate, sodium trideceth sulfate, cocamidopropyl betaine, sodium lauroamphoacetate, PEG-150 distearate, sodium laureth-13 carboxylate and octyl phenol ethoxylate (TRITON X-100). the soap can also be Coladet BSB containing water, PEG-80, sorbitan aurate, sodium trideceth sulfate, cocamnidopropyl betaine, sodium lauroamphoacetate, PEG-150 disterate, and sodium laureth-13 carboxylate.

A second water-soluble surfactant is normally present in the composition, in the range of about 0.01 to 7.0% by weight. This surfactant can be cocamidopropyl PG-dimonium chloride (COLALIPID C™), disodium lauroamphodiacetate or mixtures of the compounds.

The skin conditioning and antibacterial agent is present in a range of about 0.1 to 5.0% by weight of the composition. It can include derivatives, including quaternary compounds of phospholipid essential fatty acids (PEFA), chelating agents or mixtures thereof. For example, the skin conditioning and antibacterial agent can be linoleamidopropyl PG-diammonium chloride phosphate or a chelating agent such as disodium EDTA.

The cleanser composition also can contain a microbiological preservative system, in the range of about 0.01-11.4% by weight of the composition. This system can include preservatives that perform a variety of functions including acting as an antibacterial agent. This may include compounds such as sodium perborate; boric acid, chlorine dioxide, etidronic acid, phosphonic acid, TURPINAL SL™, phenoxyethanol, diazolidinyl urea, propylene glycol, methyl paraben, propyl paraben and their derivatives, hydrates, salts, esters and mixtures thereof.

An osmolality agent is present in the composition, in a range of about 0.1 to 6% by weight. It can be, but is not limited to, sodium chloride and potassium chloride. An aqueous solution, preferably deionized water, is present in the composition in a range of about 50 to 99.5% by weight of the composition.

Commercial Applications

The methods and compositions of the invention find numerous commercial applications that could beneficially utilize the compliance enhancing methods and compositions for eyelid hygiene. Consequently, the invention includes a kit for maintaining eyelid hygiene, e.g., treatment of an ocular disorder, in a subject. The kit includes a dispenser that is capable of generating a transiently stable foam from a cleaning agent in an aqueous solution, and instructions that set forth: (1) concentrations of eyelid cleaning agent in eyelid aqueous solution formulated to generate a transiently stable foam, (2) how to use the dispenser to generate a transiently stable foam, and (3) a method of application to maintain eyelid hygiene of a subject. The kit may further comprise 1 cleaning agent formulated to generate a transiently stable foam and/or an applicator, e.g., a sponge. The cleaning agent may be present in a pre-measured amount. The preferred dispenser is an airless foaming device, e.g., a mini airless foamer.

The kit may be used for maintaining eyelid hygiene in a subject, e.g., treatment of an ocular disorder in the subject selected from the group consisting of inflammation of tear producing glands and inflammation of the lipid producing glands. The kit comprises a controlled concentration of the cleanser composition that is formulated to generate a transiently stable foam, and a dispenser that is capable of generating the transiently stable foam from eyelid cleanser composition, e.g., an airless foaming device. The kit may optionally be packaged with instructions for use in maintaining eyelid hygiene. The cleanser composition may be present in the kit within the dispenser and/or the kit may further comprise an applicator, e.g., a sponge.

### BRIEF DESCRIPTION OF FIGURES OF THE INVENTION

FIGS. 1A and 1B depict front and side expanded views of the eyelid, respectively.

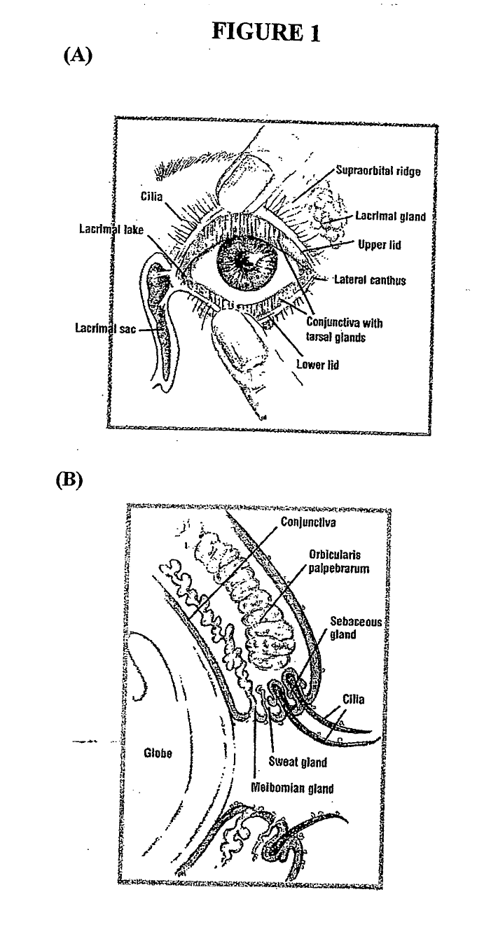

FIG. 2 depicts the entrance of bacteria through a skin portal, e.g. a follicle.

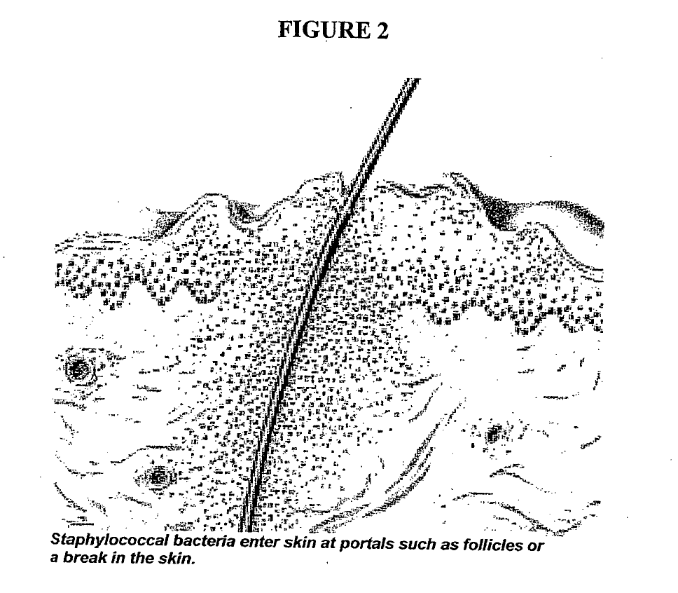

FIG. 3 depicts (a) a typical six-legged parasite present on the eyelid, (b) 3 parasitic organisms as they burrow into the eyelid, and (c) a typical eight-legged parasite present on the eyelid.

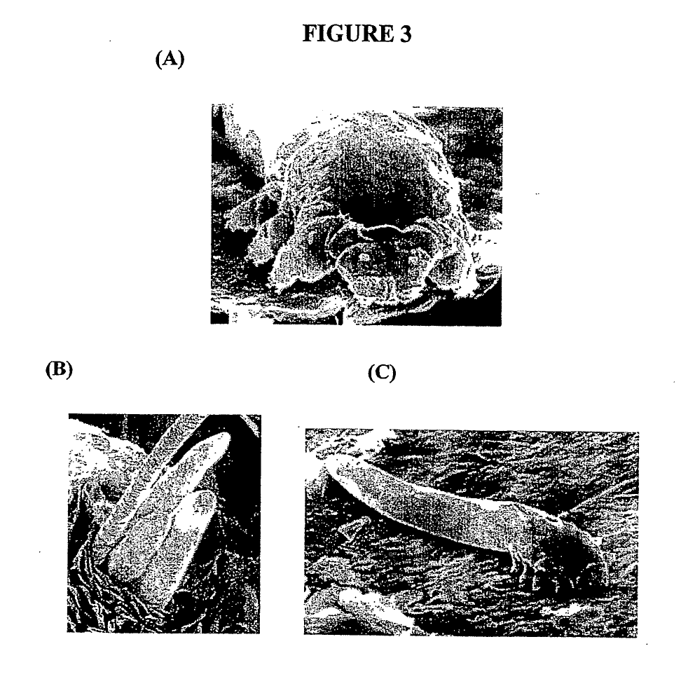

### DETAILED DESCRIPTION OF THE INVENTION

Optimization of the tear film and tear film infrastructure should include an effective method of cleaning the eyelid margin and the periocular skin. Debris causes problems not merely by blocking the tear ducts; it may also create a breeding area for pathogens close to the warm and moist environment of the eye. A method of frequently cleaning the eyelid must be easy and practical to enhance compliance.

As described above, the method of cleaning an eyelid of a subject includes the steps of providing a dispensing apparatus containing a cleanser composition and dispensing from the dispensing apparatus a controlled concentration of the cleanser composition in the form of a transiently stable foam. The dispensing apparatus can be any device that delivers a cleanser composition in the few of controlled concentration foam. However, it should be understood that, in contrast to commercially available eyelid scrubs where the sponge is stored in direct contact with the cleanser liquid, a dispenser useful in the methods of the invention is one in which the cleanser liquid, e.g. cleanser composition of the present invention, is not stored in direct contact with an applicator. For example, the dispensing apparatus may be a device that has a container portion for containing the liquid cleanser composition (or liquid cleaning agent and a separately contained aqueous portion), an induction spout that acts to thaw the liquid cleanser from the container upon actuation, and a foaming portion attached to the induction spout that creates a controlled concentration foam from the liquid composition received from the induction spout. The induction spout may be actuated by a pump or a squeeze mechanism. A preferred dispenser is an airless foamer, e.g. a mini-airless roamer.

The cleanser compositions of the invention may include any aqueous solution that contains sufficient additives, e.g., surfactants or additives with surfactant-like behavior, to produce a transiently stable foam. For example, the cleanser composition may comprise any detergent that is non-deleterious to or non-harmful on the eye (particularly at the concentrations used for maintaining eyelid hygiene). For example, baby shampoo is thought in the art to be non-irritating to the eye. The compositions of the invention include detergents that may, at certain, e.g., higher, concentrations, cause damage or discomfort to eye, but at the concentrations in the cleanser composition or foam for use in maintaining eyelid hygiene, they do not substantially interfere with the normal function of the eye. Such compositions may be prepared by standard methods known in the art of formulation.

Daily cleaning of the tear producing glands is best, especially given that the quantity and quality of performance of these glands diminish daily if untreated. As such, the cleanser composition may be formulated so that application to the eyelid does not substantially damage the skin of the eyelid, even with frequent, e.g., daily, application. Furthermore, the cleanser compositions of the invention may be formulated for any desired property, e.g., substantially non-irritating, maintenance of pH of the eyelid skin, improved ability to remove dirt and debris, and/or to increase the stability of the controlled concentration foam.

The controlled concentration foam may be prepared by generating a foam from an aqueous solution that contains sufficient additives, e.g., surfactants or additives with surfactant-like behavior, to produce a foam that is transiently stable. The controlled concentration foam provides a standardized, substantially invariable, and predefined amount of cleaning agent in a given amount of foam thus, improving the dosing regimen for maintaining eyelid hygiene. Moreover, once generated, the foam is suitable for application directly to the eyelid of subject, with the advantage that the dose of the cleaning agent is well-defined, i.e., controlled, to assist in the process of accurate prescription. This is specifically in contrast to a liquid solution that requires further preparation, e.g., dilution and/or agitation to create a foam prior to application to the eyelid. Additionally, the chosen dilution ratio may be customized based on the desired application, i.e., more concentrated for applications that require increased/enhanced cleaning.

The foam should be transiently stable in order to be useful. The foam need not be present in the form of a foam indefinitely; rather, the foam needs to be stable only as long as needed to provide a subject sufficient time to apply the dispensed foam to the eyelid. The stable foam is useful in gently removing dirt and debris from the eyelid and penetrating between the eyelashes and into the hair follicle, which are known to catch debris. Additionally, a stable foam which is applied independent of a sponge applicator contributes to the improved effectiveness of the present invention by introducing the step of massaging the eyelid with the fingers, which is more effective than the rubbing that is performed by the sponge applicators and is more beneficial for the tear glands.

Application of the foam to the eyelid of a subject may be by self-administration or by a trained professional, e.g., a doctor. More importantly, the application of the foam may be direct; e.g., it may be applied with a fingertip directly to the eyelid. In contrast to known methods of cleaning an eyelid which involve manual foaming or lathering, either with or without the agitation of a sponge, the present invention requires no additional processing or preparation of the cleanser prior to application to the eyelid. The advantage of eliminating this processing step ensures the presence of a standardized amount of cleaning agent in the resulting foam, i.e., the use of a controlled concentration foam.

The elimination of the need for applicators improves compliance by reducing the dangers associated with the applicator, such as the risk of poking or scratching of the eye with the applicator or introducing bacteria to the eye through the use of non-sterile applicators. Eliminating the applicator may have an additional economic advantage, i.e., eliminating the cost of an additional component of the treatment protocol. Moreover, with methods which require an applicator to generate a foam, the foam generation is less efficient, resulting in a reduction in the quality and amount of effective foam. However, one could apply a controlled concentration foam to an eyelid with a sponge or applicator. For example, the controlled concentration foam could be dispensed onto a sponge or applicator which would eliminate the foaming/lathering step required with known liquid cleanser compositions and ensure the delivery of a defined dosage to the eyelid.

Agitating the eyelid by localized and sustained massaging of the foam onto the eyelid improves the removal of dirt and debris from the eyelid as compared with known methods. Massaging is sustained for a period of time sufficient to substantially stimulate the cleaning of the ducts and glands in the eyelids, e.g., stimulating the removal of pooled sebum through sufficient agitation. For example, the massaging may be maintained for at least 5-30 seconds.

The method of cleaning an eyelid may further include a rinsing step. This step preferably comprises a simple water rinse. The foam may be rinsed from the eyelid with ample water after application and massage by bringing ample water to eyelid and eyelashes, e.g., with a hand, finger or any container suitable for this purpose.

The methods of the invention are not meant to only work in isolation. In this regard, it should be noted that it is within the contemplation of the present invention that the compositions and the methods may be used in conjunction with current methods in the art in a combination therapy/treatment. For example, while not appropriate for daily use, the commercially available eyelid scrubs may be used as part of a combination treatment with the compositions and methods of the present invention. The combination treatment may be utilized in any sequential arrangement, i.e., the commercially available eyelid scrubs may be used prior to, subsequent to, or interspersed with the treatment compositions and methods of the present invention More particularly, this invention provides a kit, as described above, containing a product useful for cleaning an eyelid, optionally packaged with additional instructions for use in maintaining eyelid hygiene in conjunction with the kits of the present invention.

Accordingly, the present invention provides improved methods of cleaning an eyelid, as well as methods of treatment of ocular disorders utilizing these methods, satisfying the need for a simple, easy, and compliance enhancing method of cleaning an eyelid that provides correct and defined dosage in the form of a foam. In fact, the advantages of the present invention include, but are not limited to improved removal of dirt and/or skin debris, improved removal of pooled sebum containing the toxic waste of bacteria and parasites (as wells as their eggs) by stimulation to the eyelids, non-irritating to the eyelid, reduced poking of the eye with cumbersome applicator sponges, increased compliance, daily application without substantial damage to the eyelid, and reduction in factors contributing to the creation of a breeding ground for pathogens. Moreover, the methods of the present invention incorporate cleansing, tissue repair, preventive maintenance, and treatment properties in a practical and consumer-friendly application.

### EXEMPLIFICATION OF THE INVENTION

The present invention may be further illustrated by the following non-limiting examples.

### Example 1

This example shows several formulations of a cleanser composition that can be used in the present invention. Preferably, the cleanser composition is placed in premixed form in a foaming dispenser bottle such as Minifoamer, available from Airspray International Inc. This generates the transiently stable foam that can be used in the methods of the invention.

Current research underscores that the physiological processes in and on the skin take place best in a slightly acidic medium. To support the skin's functions, it is therefore important to use skin care products with a physiological pH ideally close to the skin's pH of 5.5. In addition, it is important to stabilize the skin's natural protective acid mantle to optimize its ability to withstand infection. Accordingly the pH range of the composition is best kept about 5.5-6.5, inclusive. Citric acid is a preferred pH control agent.

The composition may include deionized water (20-80% by composition) sodium laureth sulfate in a concentration of 0.3-20%, Potassium C 12-13 monoalkyl phosphate polysorbate 60 (0.5-15%), potassium C 12-13 monoalkyl phosphate (0.5-15%), disodium lauroamphodiacetate (0.02-12%), linoleamidopropyl PG-diammonium chloride phosphate (0.01%-5.0%), sodium chloride (0.1-6%), HEPES acetate (0.01-7.7%), citric acid(0.5-7.5%) diazolidinyl urea propylene glycol/methyl-propyl parabens (0.01-5.7%), panthenol (0.01-10%), glyoxylic-diureide/allantoin (0.01-15%), polysorbate 80 (0.01-5%) and a fragrance (0.005-3%).

In one aspect, the following active ingredients are usefully combined as having proven clinical performance as an eyelid cleanser: HEPES, oleate ester as an anti-inflammatory agent (2.00 s); a pH-control agent citric acid (1.50 s); a first tissue healing agent allantoin (0.25 s); potassium C12-13 monoalkyl phosphate (a first surfactant) (5.0 s); disodium lauroamphodiacetate (a second surfactant) (4.0 s.); diazolidinyl urea propylene (a preservative) (1.00 s.); a skin conditioning and antibacterial phospholipid essential fatty acid (0.1-5.0 s.); and sodium chloride (7.00 s.). Optionally, for ascetic reasons, a natural fresh and clean fragrance may be included in this formulation.

The commercially available water-soluble surfactants useful with this invention include, but not limited to: sodium lauryl sulfate (CALFOAM ES 303); potassium C 12-13 monoalkyl phosphate 60 (ARLATONE MAP 230-T60); C12-14 monoakyl phosphate (ARLATONE MAP 230K40); disodium lauroamphodiacetate (MONATERIC 949-J); and linoleamidopropyl PG-chloride phosphate (PHOSPHOLIPID EFA). Furthermore, certain ingredients, such as allantoin, panthenol and citric acid, may be incorporated into the cleansing solution.

Examples of formulations useful in the present invention are shown below in Tables I, II, III and IV as Formulations A, B, C and D respectively.

TABLE I
Formulation A

| % | FORMULATION |
|---|---|
| 69 | Aqueous extract of chamomile |
| 14 | Potassium C-12-13 monoalkyl phosphate 80, Polysorbate 80 |
| 5 | Potassium C 12-13 monoalkyl phosphate |
| 5 | Disodium Lauroamphodiacetate |
| 4 | Sodium Borageamidopropyl hydroxyphosphate |
| 1 | Sodium chloride |
| 1 | Diazolidinyl urea |
| | Methlyparaben |
| | Propylparaben |
| 0.5 | Panthenol |
| 0.25 | Glyoxylic-Diureide/Allantoin |
| 0.1 | Polysorbate 80 (a polyoxyethylene fatty acid ester based |
| | on sorbitol) |
| 99.85 | |

TABLE II
Formulation B

| TRADE NAME | CHEMICAL NAME | FUNCTION | PREF. |
|---|---|---|---|
| DEIONIZED WATER | Deionized water | Aqueous solution | 60.50 |
| | | Aqueous with soothing | |
| | | properties | |
| CALFOAM ES 303 | Sodium Lauryl Sulfate | Surfactant cleanser | 15.00 |
| ARLATONE MAP 230- | Potassium C 12-13 | Surfactant cleanser | 5.00 |
| TWEEN 60 ™ | monoalkyl phosphate | | |
| | Polysorbate 60 | Surfactant cleanser | 7.00 |
| ARLANTONE MAP | Potassium C 12-13 | Surfactant and lipid | 4.00 |
| 230K40 | monoalkyl phosphate | replacement agent | |
| MONATERIC 949-J | Disodium | Surfactant and lipid | 1.00 |
| | Lauroamphodiacetate | replacement agent | |
| PHOSPHOLIPID EFA | Linoleamidopropyl PG- | Skin conditioning and | 2.00 |
| (essential fatty acid) | Diammonium Chloride | antibacterial agent | |
| | Phosphate | | |
| SODIUM CHLORIDE | Sodium Chloride | osmolarity controlling agent | 2.00 |
| HEPES-, ACETATE | N(2-hydroxy ethyl) | Antioxidant and anti- | 1.50 |
| ESTER | piperazine-N’-(2-propane | inflammatory | |
| | sulfonic acid), acetate salt | | |
| CITRIC ACID | Citric acid 50% Solution | Collagen building agent, | 1.00 |
| | | antioxidant and pH control | |
| | | agent | |
| GERMABEN 11 | Diazolidinyl urea | Optional microbiocidal | .50 |
| | Methyl paraben | preservative | |
| | Propyl paraben | | |
| PANTHENOL | Panthenol-alcohol analog of | Wound healer, tissue repair & | 0.25 |
| | pantothenic Acid | healing, stimulator of cellular | |
| | | proliferation, anti-inflammatory | |
| ALLANTOIN | Glyoxylic-Diureide | Stimulates new and healthy | 0.25 |
| | | tissue growth, healing | |
| | | epithelization Counter irritant, | |
| | | moisturizer, softens skins | |
| TWEEN 80 ™ | Polysorbate 80 (fatty acid | Solubilizer of optional | 0.25 |
| | ester based on sorbitol) | fragrance inclusion | |
| TOTAL | | | 100.00 |

### Example 2

### Preparation of Formulation C and Formulation D

Formulations C and D (see Table III and IV) were prepared using the following procedure. Water was added to a final mixing vessel. Slow propeller agitation was begun and the disodium EDTA and allantoin were added to the water. The components were mixed until dissolved. Hepes acetate, a solution of citric acid, boric acid, panthenol and salt were added one at a time. The solution was mixed until it was clear using slight heat as necessary. Coladet BSB™ was added, followed by Colalipid C™. The solution was mixed until it was uniform. A fragrance was premixed with Triton X 100™, then subsequently added to the batch. The solution was treated with as much 50% citric acid as necessary until a desired pH of 5.5-5.6 was attained.

TABLE III
Formulation C

| Ingredients | | Lot # | |
|---|---|---|---|
| Trade Name | CTFA Name | Manufacturer | % |
| DI Water | Deionized Water | In house | 93.68 |
| Coladet BSB | Water, PEG-80, | Colonial Chem. | 3.50 |
| | Sorbitan Laurate, | | |
| | Sodium Trideceth | | |
| | Sulfate, | | |
| | Cocamidopropyl | | |
| | Betaine, Sodium | | |
| | Lauroamphoacetate, | | |
| | PEG-150, Distearate, | | |
| | Sodium Laureth-13 | | |
| | Carboxylate | | |
| Hepes Acetate | Hepes Acetate | Hawkeye-Jensen, | 1.00 |
| | | Inc. | |
| Sodium Chloride | Sodium Chloride | Ashland | 0.85 |
| Citric acid | Citric acid | Ashland | 0.45 |
| (50% sol'n) | | | |
| Boric acid | Boric acid | Ashland Dist. Co | 0.20 |
| Panthenol | Panthenol | Ashland | 0.10 |
| Allantoin | Allantoin | Ruger Chem. Co | 0.10 |
| Colalipid C | Cocamidopropyl PG- | Colonial Chem. | 0.05 |
| | Dimonium Chloride | | |
| Sodium Perborate | Sodium Perborate | Spectrum Chemical | 0.028 |
| Monohydrate | | | |
| Turpinal SL | Etidronic acid, | Solusia | 0.006 |
| | Phosphonic acid | | |
| Disodium EDTA | Disodium EDTA | Ashland | 0.025 |
| Triton X 100 | Octoxynol-9 | Union Carbide | 0.005 |
| Fresh N' Clean | Fragrance | Bell Flavors and | 0.005 |
| J6427 | | Fragrances | |
| TOTAL | | | 100.00 |

TABLE IV
Formulation D

| Ingredients | | | |
|---|---|---|---|
| Trade Name | CTFA Name | Manufacturer | % |
| DI Water | Deionized Water | In house | 93.65 |
| Coladet BSB | Water, PEG-80, | Colonial Chem. | 3.50 |
| | Sorbitan Laurate, | | |
| | Sodium Trideceth | | |
| | Sulfate, | | |
| | Cocamidopropyl | | |
| | Betaine, Sodium | | |
| | Lauroamphoacetate, | | |
| | PEG-150, Distearate, | | |
| | Sodium Laureth-13 | | |
| | Carboxylate | | |
| Hepes Acetate | Hepes Acetate | Hawkeye-Jensen, | 1.00 |
| | | Inc. | |
| Sodium Chloride | Sodium Chloride | Ashland | 0.85 |
| Citric acid | Citric acid | Ashland | 0.45 |
| (50% sol'n) | | | |
| Boric acid | Boric acid | Ashland Dist. Co | 0.20 |
| Panthenol | Panthenol | Ashland | 0.10 |
| Allantoin | Allantoin | Ruger Chem. Co | 0.10 |
| Colalipid C | Cocamidopropyl PG- | Colonial Chem. | 0.05 |
| | Dimonium Chloride | | |
| Sodium Perborate | Sodium Perborate | Spectrum Chemical | 0.056 |
| Monohydrate | | | |
| Turpinal SL | Etidronic acid, | Solusia | 0.006 |
| | Phosphonic acid | | |
| Disodium EDTA | Disodium EDTA | Ashland | 0.025 |
| Triton X 100 | Octoxynol-9 | Union Carbide | 0.005 |
| Fresh N’ Clean | Fragrance | Bell Flavors and | 0.005 |
| J6427 | | Fragrances | |
| TOTAL | | | 100.00 |

### Example 3

### Comparative Bactericidal Study I

Organisms were isolated from cases of endophthalmitis. The organisms comprised 19 strains of S. epidermidis, 8 strains of S. aureus, 4 strains of P. aeruginosa, 2 strains of methicillin-resistant S. aureus, 3 strains of S. warneri, and 3 strains of S. marcescens. Inoculuns were prepared from overnight cultures, and 10 μl aliquots were inoculated into 1) a control tube containing 1 ml of tryptic soy broth, 2) 1 ml of EyeCl-08 and 3) 1 ml of EyeCl-12. EyeCl-08 (0.28% sodium perborate) and EyeCl-12 (0.56% sodium perborate) are formulations prepared with the sodium perborate preservative. Tubes ware incubated overnight in a non-CO₂ incubator. All of the control tubes showed bacterial growth while EyeCl-08 and EyeCl-12 tubes showed no growth. Thus, EyeCl-08 and EyeCl-12 are eyelid cleansers that are bactericidal and may help reduce the incidence of endophthalmitis.

### Example 4

### Comparative Bactericidal Study II

In another experiment, four solutions were tested in a comparative bactericidal study. Three of the formulas were prepared using different preservative agents: EyeCl-08 (0.28% sodium perborate), EyeCl-10 (0.3% Diazolidinyl urea) and EyeCl-11 (0.3% Quaternium 15). The fourth solution was a control Germaben solution.

Results are depicted in Table V. Results are the average of three zones, measured from the edge of paper disk to the edge of area on non-growth. Results are recorded in mm. The results showed that the Germaben solution did not suppress growth.

TABLE V

| | Serratia | Pseudomonas | | Staph | Moraxella |
|---|---|---|---|---|---|
| | Marcescens | aeruginosa | E. Coli | Aureus | catarrhalis |
| EyeCl-08 | 9.20* | 5.36* | 4.03* | 2.23 | 2.145 |
| EyeCl-10 | 4.025 | 3.51* | 3.00 | 2.54 | 5.855 |
| EyeCl-11 | 5.17 | 3.925 | 5.295 | 2.89 | 7.44 |
| 1.0% | 2.46 | 2.005* | 0 | 0 | 2.105* |
| Germaben | | | | | |
| II Solution | | | | | |

*Means that the zone was only suppressed growth, not complete growth.

These results show that the sodium perborate preservation system provided beneficial effects against various bacteria. In addition, there was no discomfort from the sodium perborate nor did the sodium perborate solution sting significantly.

### INCORPORATION BY REFERENCE

The entire contents of all patents, published patent applications and other references cited herein are hereby expressly incorporated herein in their entireties by reference.

Equivalents

Those skilled in the art will recognize, or be able to ascertain, using no more than routine experimentation, many equivalents to specific embodiments of the invention described specifically herein. Such equivalents are intended to be encompassed in the scope of the following claims.

1-48. (canceled)

49. A cleanser composition effective for maintaining eyelid hygiene suitable for direct application to an eyelid of a subject, comprising the following components:
(a) an anti-inflammatory agent, in the range of about 0.01 to 7.7% by weight, including zwitterionic organic compound derivatives synthesized from piperazine-N′ propane sulfonic acid;
(b) a pH-control agent and antioxidant agent, in the range of about 0.05-7.5% by weight, selected from the group consisting of a carboxylic acid derivative of propane, citric acid and mixtures thereof;
(c) a first tissue healing agent, in the range of about 0.01 to 1% by weight, selected from the group consisting of allantoin, panthenol and mixtures thereof;
(d) a soap in the form of a first water-soluble surfactant, in the range of about 0.02-20% by weight, selected from the group consisting of sodium lauryl sulfate, potassium lauryl phosphate, polyoxyethylene (20) sorbitan monostearate, polyoxyethylene sorbitan monooleate, sodium borageamidopropyl hydroxyphosphate, potassium tridecyl phosphate, potassium monoalkyl phosphate, PEG-80, sorbitan laurate, sodium trideceth sulfate, cocamidopropyl betaine, sodium lauroamphoacetate, PEG-150 distearate, sodium laureth-13 carboxylate and octyl phenol ethoxyiate;
(e) a second water soluble surfactant, in the range of about 0.01 to 7.0% by weight, selected from the group consisting of a cocamidopropyl PG dimonium chloride, disodium lauroamphodiacetate and mixtures thereof;
(f) a skin conditioning and antibacterial agent, in the range of about 0.1 to 5.0% by weight, selected from the group consisting of derivatives of phospholipid essential fatty acids, a chelating agent and mixtures thereof;
(g) a microbiological preservative system, in the range of about 0.01-11.4% by weight, selected from the group consisting of sodium perborate, boric acid, chlorine dioxide, etidronic acid, phosphoric acid, phenoxyethanol, diazolidinyl urea, propylene glycol, methyl paraben, propyl paraben and derivatives, hydrates, salts, esters and mixtures thereof.
(h) an osmolality agent, in the range of about 0.1 to 6% by weight, selected from the group consisting of sodium chloride and potassium chloride; and (i) deionized water in the range of about 50 to 99.5% by weight.

50. The cleanser composition of claim 49, wherein said microbiological preservative system includes about 0.01% to about 0.10% by weight sodium perborate and its hydrates.

51. The cleanser composition of claim 50, wherein said microbiological preservative system includes about 0.01% to about 0.06% by weight sodium perforate and its hydrates.

52. The cleanser composition of claim 50, wherein said microbiological preservative system further includes one compound selected from the group consisting of etidronic acid, phosphoric acid, boric acid and mixtures thereof.

53. The cleanser composition of claim 52, wherein said pH-control agent is citric acid and salts thereof.

54. The cleanser composition of claim 53, wherein said first surfactant is selected from the group consisting of PEG-80, sorbitan laurate, octyl phenol ethoxylate and mixtures thereof.

55. The cleanser composition of claim 54, wherein said second surfactant includes cocamidopropyl PG-dimonium chloride.

56. The cleanser composition of claim 55, wherein said osmolality agent is sodium chloride.

57. The cleanser composition of claim 56, wherein said anti-inflammatory agent is Hepes acetate.

58. The cleanser composition of claim 57, wherein said skin conditioning and antibacterial agent is a chelating agent, and wherein said chelating agent is disodium EDTA.

59. The cleanser composition of claim 49 further comprising a fragrance.

60. The cleanser composition of claim 59, wherein said fragrance is an aqueous extract of chamomile.

61. The cleanser composition of claim 49, wherein the osmolality of the solution is less than about 200 mOsmol/kg.

62. The cleanser composition of claim 61, wherein the osmolality of the solution is about 150 mOsmol/kg.

63. The cleanser composition of claim 50, wherein the osmolality of the solution is less than about 200 mOsmol/kg.

64. The cleanser composition of claim 63, wherein the osmolality of the solution is about 150 mOsmol/kg.

65. The cleanser composition of claim 49, wherein said cleanser composition is in the form of a controlled concentration foam.

66. The cleanser composition of claim 50, wherein said cleanser composition is in the form of a controlled concentration foam.

67. The cleanser composition of claim 49, wherein said first surfactant includes a compound selected from the group consisting of sodium lauryl sulfate, potassium monoalkyl phosphate, polyoxyethylene (20) sorbitan monostearate, polyoxyethylene sorbitan monooleate, sodium borageamidopropyl hydroxyphosphate and mixtures thereof.

68. The cleanser composition of claim 67, wherein said second surfactant includes disodium lauroamphodiacetate.

69. The cleanser composition of claim 68, wherein said osmolality agent is sodium chloride.

70. The cleanser composition of claim 69, wherein said anti-inflammatory agent is Hepes acetate.

71. The cleanser composition of claim 70, wherein said pH-control agent is citric I acid and salts thereof.

72. The cleanser composition of claim 71, wherein said anti-microbial preservative system comprises a compound selected from the group consisting of diazolidinyl urea, methyl paraben, propyl paraben and mixtures thereof.

73. The cleanser composition of claim 72, wherein said skin conditioning and antibacterial agent includes linoleamidopropyl PG-diammonium chloride phosphate.

74. The cleanser composition of claim 73, wherein said cleanser composition is in the form of a controlled concentration foam.

75. The cleanser composition of claim 73, further comprising a fragrance.

76. The cleanser composition of claim 75, wherein said fragrance is an aqueous extract of chamomile.
